(19) United States
(12) Patent Application Publication
(10) Pub. No.: US 2011/0290254 A1
(43) Pub. Date: Dec. 1, 2011

(54) TRACHEAL TUBE WITH CONNECTOR INSERT

(75) Inventors: Paul Waldron, Galway (IE); Brian Ledwith, Athlone (IE); Roger Harrington, Athlone (IE); Seamus Maguire, Athlone (IE)

(73) Assignee: Nellcor Puritan Bennett LLC, Boulder, CO (US)

(21) Appl. No.: 12/788,569

(22) Filed: May 27, 2010

(51) Int. Cl. A61M 16/04

(52) U.S. Cl. 128/207.14

(57) ABSTRACT

A tracheal tube assembly includes a connector body, a cannula extending from the connector body, and an insert that provides rigidity to the connector body and retains the cannula in the connector body. The cannula has a tapered upper end that fits between conforming tapered sections of the connector body inner surface and the insert. The insert may include features to prevent rotation of the cannula as well as rotation of the insert in the connector body. Features of the insert and connector body may also interface to mechanically retain the insert in the connector body. Different sizes of cannula may be accommodated by different inserts, while using the same or different connector bodies.

### BACKGROUND

The present disclosure relates to a tracheal tube, and more particularly to a tracheal tube having a connector insert for rigidifying a connection and securing a cannula to the connector.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide range of applications exist for artificial ventilation, which may call for the use of tubes that are inserted into a patient. Such tubes may include endotracheal tubes, tracheostomy tubes, and so forth. In the former case, the tubes are typically inserted through the mouth and into the trachea. In the latter, the tubes are often inserted into an opening formed in the neck and trachea of the patient. In both cases, the tubes may be used for artificial ventilation or for assisting patient ventilation. They are typically designed to interface with standard connectors that are located at the end of a ventilation hose assembly which itself may be connected to a ventilator.

Current designs for such tubes may allow for easy connection to an upper connector, but may have various structures, some quite complex, for conveying air between the connector and a cannula that extends into the patient. In some cases, a soft plastic or rubber is used for the connector, providing a nice seal with the interfacing ventilation assembly, although such soft materials may collapse or deform when pressed into the mating connector element. Moreover, difficulties exist in the mounting of the cannula in such devices, which must interface with the connector portion to provide the desired airflow path. The sizes of such cannulas may vary substantially, depending upon the anatomy of the patient, the age of a patient, and so forth. For example, the inner diameter of cannulas for pediatric and neonatal patients may vary between 2.5 mm and 6.5 mm. Larger sizes may be provided, but it would be desirable to have a uniform system of attachment between the cannula and the connector independent of the size.

### SUMMARY

The present invention provides a novel arrangement for a tracheal tube designed to respond to such needs. In accordance with one embodiment, a connector is provided having a generally annular body. A cannula has an upper end that is disposed in the annular body of the connector. An insert is disposed in the annular body of the connector and has a tubular lower extremity that contacts an upper end of the cannula to retain the cannula lodged within the annular body.

In a similar arrangement, a tracheal tube may include a connector that has a generally annular body with an outer surface dimensioned to a standard connector size. A cannula has an upper end that is disposed in the annular body in at contacts the inner surface of the annular body. An insert is disposed in the annular body and has a tubular lower extremity that contacts the upper end of the cannula to compress the cannula between the insert and the inner surface of the annular body to retain the cannula lodged within the annular body.

Also provided is a method for making a tracheal tube. The method includes inserting a cannula into a lower opening in an annular body of a connector. An insert is inserted into an upper opening of the annular body to expand and compress the upper end of the cannula against an inner surface of the connector to retain the cannula in the connector.

### BRIEF DESCRIPTION OF THE DRAWINGS

Advantages of the disclosed techniques may become apparent upon reading the following detailed description and upon reference to the drawings in which:

FIG. 1 is a perspective view of a tracheal tube in accordance with aspects of the present disclosure;

FIG. 2 is an exploded view of the same arrangement with an insert removed from the connector body;

FIG. 3 is a further exploded view of the same arrangement illustrating the connector body, the cannula prior to insertion in the connector body, and the insert shown in FIG. 2 that retains the cannula in the connector body; and

FIG. 4 is a sectional view of the same arrangement, illustrating internal features of the connector body, the insert, and the cannula when the three are joined in the completed tracheal tube.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

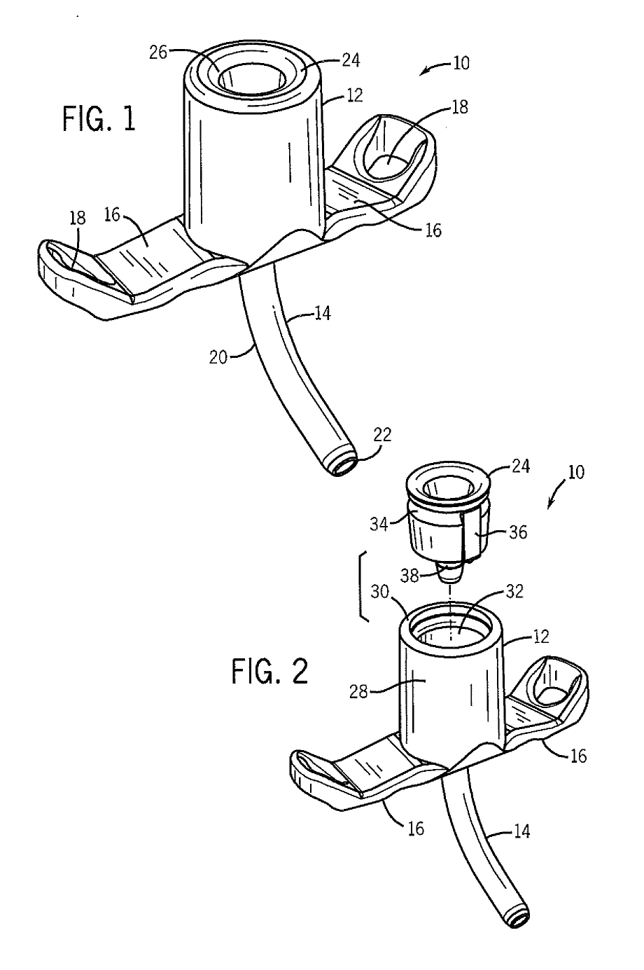

FIG. 1 illustrates an exemplary tracheal tube in accordance with the present disclosure, designated generally by reference numeral 10. In the illustrated embodiment, the tracheal tube is designed as a tracheostomy tube, although the present assembly techniques could be used in other tubes, such as endotracheal tubes and so forth. In the illustrated embodiment, the tracheal tube 10 includes a connector 12 designed to be attached to an artificial ventilation system. Various sizes of such connectors are available, and the connector may be dimensioned to accommodate any one of these sizes. In the illustrated embodiment, the connector has an outer diameter of approximately 15 mm to conform to standard dimensions of mating connectors. A cannula 14 extends from a lower end of the connector and is designed for insertion into the airway of a patient. Moreover, side flanges 16 extend from the connector for facilitating securement of the device to a patient. In the case of the tracheal tube shown, the flanges 16 may terminate in apertures 18 through which straps or other attachments devices can be inserted. The tracheal tube may be held in place on the neck of a patient by such straps. It should be noted that certain arrangements that incorporate the present teachings, such as endotracheal tubes, may not be provided with flanges.

In the illustrated embodiment, the cannula 14 is a hollow tube that can direct air or other ventilation gasses into and out of a patient. To conform more aptly to the patient anatomy, a curved section 20 may be provided as shown. The curved section ends in a lower or distal tip 22 which will be lodged in the patient during use. The illustrated cannula is not designed to be sealed to the patient airway, although the present design is intended to extend to cannulas having one or more inflatable sealing cuffs (not shown).

The connector 12 further includes an insert 24 which is disposed inside the connector body as described more fully below. As also described below, the insert serves to rigidify the connector body and to retain the cannula within the connector body. Moreover, the insert may assist in preventing rotation of the cannula within the connector body. An air passageway 26 is formed through the insert 24 and extends through the cannula such that, when coupled to appropriate ventilation devices, air or other gasses may be freely exchanged between the upper or proximate end of the connector and the distal end 22 of the cannula.

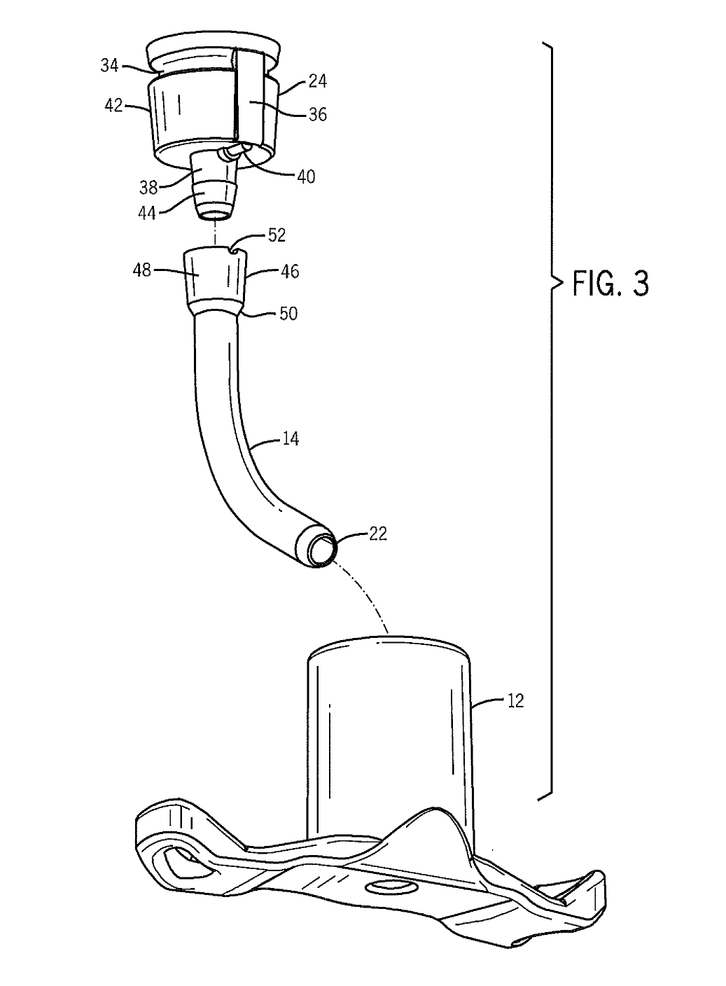

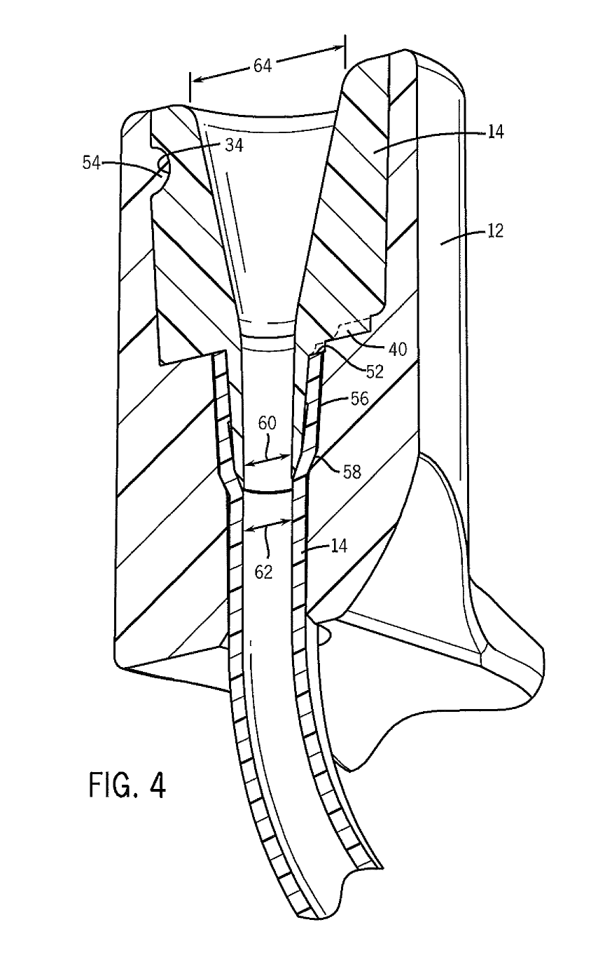

FIG. 2 shows the same arrangement with the insert 24 removed. As illustrated, the connector 12 comprises a connector body 28 which is generally annular in shape and extends upwardly from the flanges 16. The body has an upper open end 38 to form an annular structure that receives the insert 24 during assembly. The annular body 28 further has an internal surface 32 that may be provided with features for retaining the insert and for preventing rotation of both the insert and the cannula 14 as described below. In the particular embodiment illustrated, a groove 34 is provided near the upper end of the insert, and this groove will interface with a conforming feature of the connector body when inserted, as also described below. Moreover, a flat or other key structure 36 is provided to prevent rotation of the insert within the connector body. Also visible in FIG. 2 is a lower extremity 38 of the insert. As described with particular reference to FIGS. 3 and 4, this lower extremity is configured to conform to an upper end of the cannula, to lodge and compress the cannula between this lower extremity of the insert and the inner surface of the connector body.

FIG. 3 illustrates both the cannula 14 and the insert 24 exploded above the connector as they might appear prior to insertion in the connector. As illustrated, the insert 24 includes, in the present embodiment, a protrusion 40 along its lower side, in this embodiment adjacent to the flat structure 36. The insert itself has a generally tapered body 42 above the lower extremity 38. The groove 34 is positioned above this tapered body. The lower extremity 38, in turn, has a tapered portion 44 that interfaces with the cannula as described below.

The cannula itself has an upper end 46 which forms a tapered upper section 48 configured to fit against the inner surface of the connector body as described below. A lower tapered section 50 also contacts and interfaces with the inner surface of the connector body to retain the cannula in place of within the connector. In the illustrated embodiment, a channel or notch 52 is formed in the upper end of the cannula that receives the protrusion 40 in the insert when the pieces are assembled within the connector. The fit of the protrusion within the channel precludes a rotational movement of a cannula within the connector. The three components will typically be formed separately. The connector and insert will typically be molded, while the cannula may be made by an extrusion process. Other processes may, of course, be employed where desired and appropriate. In a presently contemplated embodiment, the connector is made of a soft polyvinylchloride or other synthetic plastic. The soft material of the connector allows for easy gripping and a good contact fit with the mating connector part when the tube is connected to a ventilation system. The softer material also allows for comfort against the patient's neck when the device is not connected to a mating connector (e.g., of a ventilation system). The flanges may also be molded with the body of the connector, or these could be added in a separate operation. In a presently contemplated embodiment, the flanges are co-molded or over-molded with the connector body. The cannula 14 may also be made of a synthetic plastic material, such as soft polyvinylchloride, polyurethane, thermoplastic elastomers, or other synthetic plastics. The insert 24 may be made of a harder material than the connector body, such as a hard polyvinylchloride, a polycarbonate plastic, ABS, or any suitable material or a combination of materials. Where the insert is harder than the soft connector body, it provides rigidity to a connector body and resists forces that might tend to collapse the connector body, such as from mating connectors, and so forth. The more rigid structure also provides a good surface to which the cannula may be bonded, and that supports the inner diameter of the cannula.

The assembly may be performed by first inserting the insert into the cannula, then by inserting both the cannula and the insert into the connector, such that the cannula lower end extends through the connector and the cannula seats within the connector as described below. During insertion, the insert is aligned with the connector inner surface and pressed into place. In the present embodiment, the retention features of the insert cooperating with those of the connector prevent the insert from being easily removed from the connector. Although mechanical features are built into the connector and insert in the embodiment illustrated, such mechanical features may be complimented by various bonding agents and/or adhesives. In certain embodiments, the insert and connector body may be co-molded or over-molded. It should be noted that the assembly may proceed in different orders (e.g., by insertion of the cannula in the connector with or separately from the insert), depending upon the particular configuration of the components, the nature of the retaining features of each, and the type of processes used for formation and assembly (e.g., bonding, overmolding, etc.).

FIG. 4 illustrates the three components of the tracheal tube in section. As described above, when assembled, the cannula 14 is lodged within the connector body, and retained in place by the insert 14. Moreover, the insert 14 prevents rotation of the cannula by the lower protrusion 40 that enters within the channel 52 formed in the upper end of the cannula. Similarly, the flat structure 36 illustrated in FIG. 3 cooperates with a similar surface of the connector body to prevent rotation of the insert within the connector body. In the arrangement shown in FIG. 4, moreover, an inner protrusion 54 of the connector body enters into groove 34 of the insert to mechanically retain the insert within the connector body. Here again, glues and bonding agents may also be employed to retain these components in the assembled positions shown in FIG. 4. Still further, the upper tapered section 48 of the cannula (see FIG. 3) is configured to conform to a similar upper tapered inner wall section 56 of the connector body. The lower tapered section 50 of the cannula upper end (see FIG. 3) similarly conforms generally to a lower tapered inner wall section 58 of the connector body. Thus, the insert, which fits within the upper end of the cannula, tends to expand or compress the upper end of the cannula slightly against the inner surface of the connector body. This cooperation retains the cannula within the connector body, and prevents rotation of the cannula with the connector body.

It should be noted that the sizes of these components may be adapted to conform to various standard sizes of tracheal tubes. For example, in tubes used for pediatric and neonatal patients, an inner diameter of the cannula may vary between 2.5 and 6.5 mm. Other sizes, could, of course, be accommodated. It should also be noted that, as shown in FIG. 4, the inner diameter of the lower extremity of the insert, indicated by reference numeral 60, will generally conform to the inner diameter 62 of the cannula. This arrangement allows for the easy passage of air or other ventilation gasses without creating an obstruction either in the connector or the cannula. The upper end of the opening and the insert, indicated by reference numeral 64, preferably expands to allow for the channeling of air or ventilation gasses easily into the assembly. It should also be noted that a range of sizes of inserts may be accommodated for the same external dimension of the connector body. Thus, various sizes of inserts 14 may be designed to interface with various sizes of cannulas. This may be done while maintaining the configuration and even the size of the connector body the same. Thus, the same connector body may be used with different inserts and cannula sizes to obtain a family of tracheal tubes.

What is claimed is:

1. A tracheal tube comprising:
a connector having a generally annular body;
a cannula having an upper end disposed in the annular body; and
an insert disposed in the annular body and having a tubular lower extremity contacting the upper end of the cannula to retain the cannula lodged within the annular body.

2. The tracheal tube of claim 1, wherein the tubular lower extremity of the insert has an inner diameter approximately equal to an inner diameter of the cannula.

3. The tracheal tube of claim 1, wherein the annular body comprises an anti-rotation feature that interfaces with the insert to prevent rotation of the insert within the annular body.

4. The tracheal tube of claim 1, wherein the annular body comprises a retaining feature that interfaces with the insert to prevent removal of the insert from the annular body.

5. The tracheal tube of claim 1, wherein the annular body comprises a pair of laterally extending flanges for facilitating securement to a patient.

6. The tracheal tube of claim 1, wherein the lower extremity of the insert is tapered to expand the upper end of the cannula towards an inner surface of the connector.

7. The tracheal tube of claim 1, wherein the annular body is made of an elastomeric material, and the insert is made of a material harder than the elastomeric material of the annular body.

8. The tracheal tube of claim 1, wherein the insert had an inner surface tapered from a first diameter within the insert to a second larger diameter at an upper end of the insert.

9. A tracheal tube comprising:
a connector having a generally annular body with an outer surface dimensioned to a standard connector size;
cannula having an upper end disposed in the annular body and contracting an inner surface of the annular body; and
an insert disposed in the annular body and having a tubular lower extremity contacting the upper end of the cannula to compress the cannula between the insert and the inner surface of the annular body to retain the cannula lodged within the annular body.

10. The tracheal tube of claim 9, wherein at least a portion of the lower extremity of the insert is tapered to expand the upper end of the cannula towards the inner surface of the annular body.

11. The tracheal tube of claim 10, wherein an inner surface of the annular body is tapered in a region facing the tapered portion of the lower extremity of the insert to deform the cannula outwardly.

12. The tracheal tube of claim 9, wherein the tubular lower extremity of the insert has an inner diameter approximately equal to an inner diameter of the cannula.

13. The tracheal tube of claim 9, wherein the annular body comprises an anti-rotation feature that interfaces with the insert to prevent rotation of the insert within the annular body.

14. The tracheal tube of claim 9, wherein the annular body comprises a retaining feature that interfaces with the insert to prevent removal of the insert from the annular body.

15. The tracheal tube of claim 9, wherein the annular body is made of an elastomeric material, and the insert is made of a material harder than the elastomeric material of the annular body.

16. A method for making a tracheal tube, comprising:
inserting an insert into a cannula; and
inserting the insert and cannula into an upper opening of an annular body of a connector to expand and compress an upper end of the cannula against an inner surface of the connector to retain the cannula in the connector.

17. The method of claim 16, comprising expanding the upper end of the cannula by insertion of a tapered portion of the insert into the cannula.

18. The method of claim 16, wherein the annular body is made of an elastomeric material, and the insert is made of a material harder than the elastomeric material of the annular body.

19. The method of claim 16, comprising engaging anti-rotation features of the connector and the insert to prevent rotation of the insert in the connector.

20. The method of claim 16, comprising engaging retaining features of the connector and the insert to prevent removal of the insert from the connector.
